(19) United States
(12) Patent Application Publication
(10) Pub. No.: US 2025/0170295 A1
(43) Pub. Date: May 29, 2025

(54) CONTACT LENS AND CONTACT LENS PRODUCT

(71) Applicants: LARGAN MEDICAL CO., LTD., Taichung City (TW); LE Biomedical Corp., Taoyuan City (TW)

(72) Inventors: Wei-Yuan CHEN, Taichung City (TW); Yi Shyang HUANG, Taoyuan City (TW); Yu-Yi SHIH, Taichung City (TW); Wei-Chun CHEN, Taichung City (TW); Yu Yin NI, Taoyuan City (TW); Yu Jie HONG, Taichung City (TW); Chun-Hung TENG, Taichung City (TW)

(21) Appl. No.: 18/919,502

(22) Filed: Oct. 18, 2024

(30) Foreign Application Priority Data
Nov. 28, 2023 (TW) 112146054

(51) Int. Cl. A61L 12/14; G02C 7/04

(52) U.S. Cl. CPC A61L 12/14; G02C 7/04

(57) ABSTRACT

A contact lens includes a composition of the contact lens. The composition of the contact lens includes a humectant. The humectant includes a glucan, and the glucan in the composition of the contact lens includes an 1-3/1-6-glucan. The contact lens product includes the contact lens and the buffer solution, and the contact lens is immersed in the buffer solution.

### RELATED APPLICATIONS

This application claims priority to Taiwan Application Serial Number 112146054, filed Nov. 28, 2023, which is herein incorporated by reference in its entirety.

### BACKGROUND

### Technical Field

The present disclosure relates to a contact lens and a contact lens product. More particularly, the present disclosure relates to a contact lens and a contact lens product with an excellent moisturizing property.

### Description of Related Art

The contact lenses have different fixed water contents depending on their material compositions. When the user wears the contact lenses, the water on the surface of the eyeball will be absorbed by the contact lenses to replenish the water lost from the contact lenses into the air. The loss of water will increase with the wearing time. Thus, when the user wears the contact lenses for a long time, the uncomfortable symptoms such as redness, swelling and heat are caused due to the dry eyes. In order to improve the comfort of the user wearing the contact lens for a long time and increase the willingness of the user to purchase the contact lens product, the loss of water in the contact lenses must be effectively reduced.

### SUMMARY

According to one aspect of the present disclosure, a contact lens includes a composition of the contact lens, and the composition of the contact lens includes a humectant. The humectant includes a glucan, and the glucan in the composition of the contact lens includes an 1-3/1-6-glucan. A weight percentage of the glucan in the composition of the contact lens is Pc, an average transmittance of the contact lens in a wavelength range from 400 nm to 700 nm is T4070, and the following conditions are satisfied: 0.001%≤Pc≤1.00%; and 90%≤T4070.

According to another aspect of the present disclosure, a contact lens product includes the contact lens according to the foregoing aspect and a buffer solution. The contact lens is immersed in the buffer solution.

According to further another aspect of the present disclosure, a contact lens includes a composition of the contact lens, and the composition of the contact lens includes a humectant. The humectant includes a glucan, and the glucan in the composition of the contact lens includes a beta-glucan. A weight percentage of the glucan in the composition of the contact lens is Pc, an average transmittance of the contact lens in a wavelength range from 450 nm to 630 nm is T4563, and the following conditions are satisfied: 0.0001%≤Pc; and 90%≤T4563.

According to still another aspect of the present disclosure, a contact lens product includes the contact lens according to the foregoing aspect.

According to yet another aspect of the present disclosure, a contact lens product includes a contact lens and a buffer solution. The contact lens is immersed in the buffer solution. The buffer solution includes a humectant, the humectant in the buffer solution includes a glucan, and the glucan in the buffer solution includes a beta-glucan. A weight percentage of the glucan in the buffer solution is Pb, and the following condition is satisfied: 0.0001%≤Pb≤5.00%.

### DETAILED DESCRIPTION

The present disclosure provides a contact lens including a composition of the contact lens, and the composition of the contact lens includes a humectant. The humectant includes a glucan, and the glucan in the composition of the contact lens includes an 1-3/1-6-glucan. Therefore, the glucan with the high moisturizing effect is used as the humectant for the contact lens to reduce the loss of water from the contact lens into the air, and it is favorable for improving the moisturizing property of the contact lens. Further, the preservation of the water in a lingzhi is achieved by the 1-3/1-6-glucan with the high moisturizing, and thus the 1-3/1-6-glucan which is abundant in the lingzhi is chosen as the humectant for the contact lens, so that it is favorable for maximizing the moisturizing property of the contact lens.

The present disclosure further provides a contact lens including a composition of the contact lens, and the composition of the contact lens includes a humectant. The humectant includes a glucan, and the glucan in the composition of the contact lens includes a beta-glucan. Therefore, the glucan with the high moisturizing effect is used as the humectant for the contact lens to reduce the loss of water from the contact lens into the air, and it is favorable for improving the moisturizing property of the contact lens. Further, the beta-glucan is chosen as the humectant for the contact lens, and thus it is favorable for improving the moisturizing property of the contact lens.

According to the contact lens of the present disclosure, when a weight percentage of the glucan in the composition of the contact lens is Pc, the following condition can be satisfied: 0.001%≤Pc≤1.00%. Therefore, the contact lens has both the high moisturizing and the high transmittance of the visible light by limiting the weight percentage of the glucan in the composition of the contact lens. Furthermore, the following condition can be satisfied: 0.0001%≤Pc≤10.00%. Furthermore, the following condition can be satisfied: 0.0005%≤Pc≤5.00%. Furthermore, the following condition can be satisfied: 0.003%≤Pc≤0.10%. Furthermore, the following condition can be satisfied: 0.005%≤Pc≤0.05%. Furthermore, the following condition can be satisfied: 0.006%≤Pc≤0.02%. Furthermore, the following condition can be satisfied: 0.008%≤Pc≤0.013%. Furthermore, the following condition can be satisfied: 0.0001%≤Pc.

According to the contact lens of the present disclosure, when an average transmittance of the contact lens in a wavelength range from 400 nm to 700 nm is T4070, the following condition can be satisfied: 90%≤T4070. Therefore, the contact lens can maintain the high transmittance of the overall visible light by satisfying the average transmittance in the wavelength range from 400 nm to 700 nm. Furthermore, the following condition can be satisfied: 90.5%≤T4070. Furthermore, the following condition can be satisfied: 91%≤T4070. Furthermore, the following condition can be satisfied: 91.5%≤T4070. Furthermore, the following condition can be satisfied: 92%≤T4070. Furthermore, the following condition can be satisfied: 92.5%≤T4070≤100%.

According to the contact lens of the present disclosure, when an average transmittance of the contact lens in a wavelength range from 450 nm to 630 nm is T4563, the following condition can be satisfied: 90%≤T4563. Therefore, the contact lens can maintain the high transmittance of the visible light in a specific wavelength band by satisfying the average transmittance in the wavelength range from 450 nm to 630 nm. Furthermore, the following condition can be satisfied: 90.5%≤T4563. Furthermore, the following condition can be satisfied: 91%≤T4563. Furthermore, the following condition can be satisfied: 91.5%≤T4563. Furthermore, the following condition can be satisfied: 92%≤T4563. Furthermore, the following condition can be satisfied: 92.5%≤T4563≤100%.

According to the contact lens of the present disclosure, when a molecular weight of the glucan in the composition of the contact lens is MWc, the following condition can be satisfied: 10 kDa≤MWc≤2000 kDa. Therefore, the interference of the transmittance of the visible light by the glucan can be prevented and the physical strength of the contact lens can be improved by adding the glucan with an appropriate molecular weight in the contact lens, and it is favorable for maintaining the transmittance effect of the contact lens and facilitating the structural stability of the contact lens. Furthermore, the following condition can be satisfied: 30 kDa≤MWc≤1800 kDa. Furthermore, the following condition can be satisfied: 50 kDa≤MWc≤1500 kDa. Furthermore, the following condition can be satisfied: 100 kDa≤MWc≤1000 kDa. Furthermore, the following condition can be satisfied: 150 kDa≤MWc≤700 kDa.

According to the contact lens of the present disclosure, when a particle size of the glucan in the composition of the contact lens is PSc, the following condition can be satisfied: 50 nm≤PSc≤5000 nm. Therefore, the glucan is effectively embedded in the polymerized network structure and the loss of glucan can be reduced by adding the glucan with an appropriate particle size in the contact lens, and it is favorable for improving the moisturizing timeliness of the contact lens. Furthermore, the following condition can be satisfied: 500 nm≤PSc≤4000 nm. Furthermore, the following condition can be satisfied: 1000 nm≤PSc≤3000 nm. Furthermore, the following condition can be satisfied: 1200 nm≤PSc≤2500 nm. Furthermore, the following condition can be satisfied: 1500 nm≤PSc≤2300 nm. Furthermore, the following condition can be satisfied: 1600 nm≤PSc≤2100 nm.

According to the contact lens of the present disclosure, when a diameter of the contact lens is Dc, the following condition can be satisfied: 13.8 mm≤Dc≤14.5 mm. Therefore, the lens drift caused by too small diameter and the corneal deformation caused by too large diameter can be avoided by designing an appropriate diameter of the contact lens, and it is favorable for improving the waring stability of the user. Furthermore, the following condition can be satisfied: 13 mm≤Dc≤15 mm. Furthermore, the following condition can be satisfied: 13.5 mm≤Dc≤14.8 mm. Furthermore, the following condition can be satisfied: 14.0 mm≤Dc≤14.2 mm.

According to the contact lens of the present disclosure, the beta-glucan in the composition of the contact lens can include an 1-3/1-6-glucan. Further, the preservation of the water in a lingzhi is achieved by the 1-3/1-6-glucan with the high moisturizing, and thus the 1-3/1-6-glucan which is abundant in the lingzhi is chosen as the humectant for the contact lens, so that it is favorable for maximizing the moisturizing property of the contact lens.

According to the contact lens of the present disclosure, the contact lens can be a hydrogel contact lens, and a main monomer of the contact lens can be hydroxyethyl methacrylate. Therefore, the glucan is used in the hydrogel contact lens that the main monomer is the hydroxyethyl methacrylate, and thus it is favorable for improving the wearing comfort of the hydrogel contact lens.

According to the contact lens of the present disclosure, the composition of the contact lens can further include an UV absorber or a blue light absorber, a dye, a beneficial agent, an antioxidant and a myopia control agent. Therefore, by adding the UV absorber, the blue light absorber, the beneficial agent and the antioxidant, the adverse effects such as the eye damage and the inflammation can be reduced, and the discomfort caused by the contact lens absorbing the water from the surface of the eyeball can be reduced. Further, the aesthetic of the contact lens can be increased by adding the dye, thereby increasing the willingness of the user to wear the contact lens. Further, the contact lens has the effect of controlling and delaying the progression of the myopia by adding the myopia control agent, thereby increasing the scope of application of the contact lens.

Each technical feature in the abovementioned contact lens can be configured in combination to achieve the corresponding effect.

The present disclosure further provides a contact lens product which includes the abovementioned contact lens.

The present disclosure further provides a contact lens product which includes the abovementioned contact lens and a buffer solution, and the contact lens is immersed in the buffer solution.

According to the contact lens product of the present disclosure, the buffer solution can include a humectant, the humectant in the buffer solution can include a glucan, and the glucan in the buffer solution can include a beta-glucan. Therefore, the glucan with the high moisturizing effect is used as the humectant for the buffer solution, and it is favorable for improving the wearing comfort of the contact lens. The beta-glucan is chosen as the humectant for the buffer solution, and it is favorable for further improving the wearing comfort of the contact lens.

According to the contact lens product of the present disclosure, when a weight percentage of the glucan in the buffer solution is Pb, the following condition can be satisfied: 0.0003%≤Pb≤1.00%. Therefore, the foreign body sensation can be reduced when the user places the contact lens on the eyes thereof by limiting the weight percentage of the glucan in the buffer solution. Furthermore, the following condition can be satisfied: 0.0001%≤Pb≤5.00%. Furthermore, the following condition can be satisfied: 0.0005%≤Pb≤0.50%. Furthermore, the following condition can be satisfied: 0.001%≤Pb≤0.30%. Furthermore, the following condition can be satisfied: 0.003%≤Pb≤0.10%. Furthermore, the following condition can be satisfied: 0.005%≤Pb≤0.05%. Furthermore, the following condition can be satisfied: 0.0055%≤Pb≤0.008%.

According to the contact lens product of the present disclosure, when a molecular weight of the glucan in the buffer solution is MWb, the following condition can be satisfied: 50 kDa≤MWb≤1500 kDa. The molecular weight of the glucan will affect the effectiveness of the glucan as the humectant, and by adding the glucan with an appropriate molecular weight in the buffer solution, the comfort and the foreign body sensation can be balanced while wearing the contact lens. Furthermore, the following condition can be satisfied: 10 kDa≤MWb≤2000 kDa. Furthermore, the following condition can be satisfied: 30 kDa≤MWb≤1800 kDa. Furthermore, the following condition can be satisfied: 100 kDa≤MWb≤1000 kDa. Furthermore, the following condition can be satisfied: 150 kDa≤MWb≤700 kDa.

According to the contact lens product of the present disclosure, when a particle size of the glucan in the buffer solution is PSb, the following condition can be satisfied: 500 nm≤PSb≤4000 nm. Therefore, the glucan is dispersed evenly in the buffer solution by adding the glucan with an appropriate particle size in the buffer solution, and it is favorable for improving the uniformity of the buffer solution. Furthermore, the following condition can be satisfied: 50 nm≤PSb≤5000 nm. Furthermore, the following condition can be satisfied: 1000 nm≤PSb≤3000 nm. Furthermore, the following condition can be satisfied: 1200 nm≤PSb≤2500 nm. Furthermore, the following condition can be satisfied: 1500 nm≤PSb≤2300 nm. Furthermore, the following condition can be satisfied: 1600 nm≤PSb≤2100 nm.

According to the contact lens product of the present disclosure, when a water content of the contact lens is Cw, the following condition can be satisfied: 54.5%≤Cw. Therefore, the wearing comfort and the moisturizing effect of the contact lens can be balanced by designing an appropriate water content of the contact lens. Furthermore, the following condition can be satisfied: 55.0%≤Cw≤100%. Furthermore, the following condition can be satisfied: 55.5%≤Cw≤60.0%.

According to the contact lens product of the present disclosure, the beta-glucan in the buffer solution can include an 1-3/1-6-glucan. Further, the preservation of the water in the lingzhi is achieved by the 1-3/1-6-glucan with the high moisturizing, and thus the 1-3/1-6-glucan which is abundant in the lingzhi is chosen as the humectant for the buffer solution, so that it is favorable for achieving the optimization of the wearing comfort of the contact lens.

According to the contact lens product of the present disclosure, a composition of the contact lens can include a humectant, and the humectant in the composition of the contact lens can include a beta-glucan. The beta-glucan is chosen as the humectant for the contact lens, and it is favorable for further improving the moisturizing property of the contact lens.

According to the contact lens product of the present disclosure, when a viscosity of the buffer solution is VI, the following condition can be satisfied: VI≤10 Pa·s. Therefore, it is favorable for preventing the eyes from difficulty blinking caused by the buffer solution on the surface of the contact lens by designing an appropriate viscosity of the buffer solution, so that the discomfort of the user can be reduced. Furthermore, the following condition can be satisfied: 0 Pa·s<VI≤5 Pa·s. Furthermore, the following condition can be satisfied: 0.1 Pa·s≤VI≤3 Pa·s. Furthermore, the following condition can be satisfied: 0.3 Pa·s≤VI≤2 Pa·s. Furthermore, the following condition can be satisfied: 0.5 Pa·s≤VI≤1.5 Pa·s. Furthermore, the following condition can be satisfied: 0.75 Pa·s≤VI≤1.25 Pa·s. Furthermore, the following condition can be satisfied: 0.9 Pa·s≤VI≤1.1 Pa·s.

According to the contact lens product of the present disclosure, when a pH value of the buffer solution is pHb, the following condition can be satisfied: 7.3≤pHb≤7.5. The pH value of the tears is 7.4. Therefore, the acid-base neutralization between the buffer solution on the surface of the contact lens and the tears can be avoided by designing the buffer solution with the pH value similar to that of the tears, and it is favorable for avoiding the adverse reaction. Furthermore, the following condition can be satisfied: 6.5≤pHb≤8. Furthermore, the following condition can be satisfied: 6.8≤pHb≤7.8. Furthermore, the following condition can be satisfied: 7.0≤pHb≤7.6. Furthermore, the following condition can be satisfied: 7.35≤pHb≤7.45.

According to the contact lens product of the present disclosure, when an osmotic pressure of the buffer solution is Ob, the following condition can be satisfied: 0.280 Osmol/kg·H₂O≤Ob≤0.350 Osmol/kg·H₂O. Therefore, it is favorable for preventing the epidermis of the cornea from dehydrating and swelling caused by the buffer solution on the surface of the contact lens by designing an appropriate osmotic pressure of the buffer solution, and thus the discomfort of the user can be reduced. Furthermore, the following condition can be satisfied: 0.285 Osmol/kg·H₂O≤Ob≤0.340 Osmol/kg·H₂O. Furthermore, the following condition can be satisfied: 0.290 Osmol/kg·H₂O≤Ob≤0.320 Osmol/kg·H₂O. Furthermore, the following condition can be satisfied: 0.295 Osmol/kg·H₂O≤Ob≤0.305 Osmol/kg·H₂O.

Each technical feature in the abovementioned contact lens and contact lens product can be configured in combination to achieve the corresponding effect.

The composition of the contact lens of the present disclosure can include at least one, at least two, at least three or at least four humectants. The composition of the contact lens can include at least one, at least two, at least three or at least four monomers. The composition of the contact lens can include at least one, at least two, at least three or at least four initiators. The composition of the contact lens can include at least one, at least two, at least three or at least four cross-linking agents. The composition of the contact lens can include at least one, at least two, at least three or at least four diluents. The composition of the contact lens can selectively include at least one, at least two, at least three or at least four UV absorbers or blue light absorbers. The composition of the contact lens can include at least one, at least two, at least three or at least four dyes. The composition of the contact lens can include at least one, at least two, at least three or at least four beneficial agents. The composition of the contact lens can include at least one, at least two, at least three or at least four antioxidants. The composition of the contact lens can include at least one, at least two, at least three or at least four myopia control agents. The composition of the contact lens can be located inside the contact lens or on the surface of the contact lens. When the composition of the contact lens is located inside the contact lens, the composition of the contact lens is used as the material and then cured to form the contact lens. When the composition of the contact lens is located on the surface of the contact lens, the composition of the contact lens is bonded or attached to the surface of the contact lens by coating or soaking.

The weight percentage of the composition of the contact lens of the present disclosure means the weight percentage of various components in the dry contact lens after the main body of the contact lens in the contact lens product is taken out of the buffer solution and completely dehydrated.

The humectant in the composition of the contact lens of the present disclosure can include the glucan, PEG-40 hydrogenated castor oil, PEG-400, glycerin, allantoin, hyaluronic acid, ceramide, cholesterol, sea buckthorn fruit oil, γ-polyglutamic acid (γ-PGA), trehalose, alginic acid, hydroxypropyl methylcellulose (HPMC), 2-methacryloyloxyphosphorylcholine (MPC), etc., and the salts thereof.

The three-dimensional configuration of the glucan in the composition of the contact lens of the present disclosure can be a D-form or an L-form. The glucan can include an alpha-glucan and a beta-glucan. The alpha-glucan can include alpha-1-3-glucan, alpha-1-4-glucan, alpha-1-6-glucan, alpha-1-3/1-4-glucan, alpha-1-4/1-6-glucan and alpha-1-3/1-6-glucan. The main chain of the alpha-glucan can be 1,3 bonded, 1,4-bonded or 1,6-bonded, and the branch chain of the alpha-glucan can be 1,3-bonded, 1,4-bonded or 1,6-bonded. The beta-glucan can include beta-1-3-glucan, beta-1-4-glucan, beta-1-6-glucan, beta-1-3/1-4-glucan, beta-1-4/1-6-glucan and beta-1-3/1-6-glucan. The main chain of the beta-glucan can be 1,3 bonded, 1,4-bonded or 1,6-bonded, and the branch chain of the beta-glucan can be 1,3-bonded, 1,4-bonded or 1,6-bonded. The glucan in the composition of the contact lens can further include a beta-1-3/1-6-D-glucan in the polysaccharide of the lingzhi. The main chain of the beta-1-3/1-6-D-glucan is 1,3 bonded, and the branch chain of the beta-1-3/1-6-D-glucan is 1,6-bonded. The extraction source of the glucan in the composition of the contact lens can be from the yeast, the cereal, the fungus, the plant, the bacteria, the algae, the lichen, etc. The extraction source being the yeast of the glucan in the composition of the contact lens can be baker's yeast and brewer's yeast. The extraction source being the cereal of the glucan in the composition of the contact lens can be the barley, the oatmeal, the wheat or the rice. The extraction source being the fungus of the glucan in the composition of the contact lens can be the lingzhi, the white mushroom, the maitake mushroom, the mushroom and Aspergillus niger. The extraction source being the plant of the glucan in the composition of the contact lens can be Aloe vera and Echinacea sp.

The molecular weight of the glucan in the composition of the contact lens of the present disclosure means the number average molecular weight of the overall glucan in the contact lens, and the formula thereof is: (2 molecular weight of the glucan in the composition of the contact lens)/the total number of molecule.

The particle size of the glucan in the composition of the contact lens of the present disclosure means the median of the molecular diameter of the overall glucan in the composition of the contact lens.

The type of the contact lens of the present disclosure can be a hydrogel contact lens or a silicone hydrogel contact lens. The hydrogel contact lens contains the hydrogel monomer. The silicone hydrogel contact lens contains the silicone hydrogel monomer. The main monomer of the contact lens means the monomer component with the highest weight percentage in the contact lens.

The hydrogel monomer of the contact lens of the present disclosure can include 2-hydroxyethyl methacrylate (HEMA), methacrylic acid (MAA), glycerol monomethacrylate (GMA), N-vinyl-2-pyrrolidinone (NVP), methyl methacrylate (MMA), N,N-dimethyl acrylamide (DMAA), glucan, etc.

The hydrogel of the contact lens of the present disclosure can be but not limited to the contact lens material classified as Group 1 by U.S. Food and Drug Administration (USFDA), that is, the nonionic polymer with the low water content (less than 50 weight percentage), such as Helfilcon A&B, Hioxifilcon B, Mafilcon, Polymacon, Tefilcon, Tetrafilcon A, etc. Furthermore, the abovementioned hydrogel can be the contact lens material classified as Group 2 by U.S. Food and Drug Administration, that is, the nonionic polymer with the high water content (greater than 50 weight percentage), such as Acofilcon A, Alfafilcon A, Hilafilcon B, Hioxifilcon A, Hioxifilcon B, Hioxifilcon D, Nelfilcon A, Nesofilcon A, Omafilcon A, Samfilcon A, etc. Furthermore, the abovementioned hydrogel can be the contact lens material classified as Group 3 by U.S. Food and Drug Administration, that is, the ionic polymer with the low water content (less than 50 weight percentage), such as Deltafilcon A, etc. Furthermore, the abovementioned hydrogel can be the contact lens material classified as Group 4 by U.S. Food and Drug Administration, that is, the ionic polymer with the high water content (greater than 50 weight percentage), such as Etafilcon A, Focofilcon A, Methafilcon A, Methafilcon B, Ocufilcon A, Ocufilcon B, Ocufilcon C, Ocufilcon D, Ocufilcon E, Phemfilcon A, Vifilcon A, etc.

The silicone hydrogel monomer of the contact lens of the present disclosure can be but not limited to 2-hydroxyethyl methacrylate, glycerol monomethacrylate, methacrylic acid, 3-methacryloyloxypropyltris(trimethylsilyloxy) silane, N-vinyl-2-pyrrolidinone, N,N-dimethyl acrylamide, 3-(3-methacryloxy-2-hydroxypropoxy)propyl bis(trimethylsiloxy)methylsilane, or (3-acryloxy-2-hydroxypropoxypropyl) terminated polydimethylsiloxane.

The silicone hydrogel of the contact lens of the present disclosure can be but not limited to the contact lens material classified as Group 5 by U.S. Food and Drug Administration, such as Balafilcon A, Comfilcon A, Efrofilcon A, Enfilcon A, Galyfilcon A, Lotrafilcon A, Lotrafilcon B, Narafilcon A, Narafilcon B, Senofilcon A, Delefilcon A, Somofilcon A, etc.

The initiator of the contact lens of the present disclosure can be but not limited to 2-hydroxy-2-methyl-propiophenone.

The cross-linking agent of the contact lens of the present disclosure can be but not limited to ethylene glycol dimethacrylate or 1,1,1-trimethylol propane trimethacrylate.

The diluent of the contact lens of the present disclosure can be polyethylene glycol 300, polyethylene glycol 600, polyethylene glycol 800, polyethylene glycol 1000, polyethylene glycol 2000, polyethylene glycol 4000, 1,4-butanediol, ethanol, isopropyl alcohol, glycerol or 1-hexanol.

The UV absorber of the contact lens of the present disclosure can be but not limited to 2-[3-(2H-benzotriazol-2-yl)-4-hydroxyphenyl]ethyl methacrylate, 4-methacryloxy-2-hydroxybenzophenone, 2-phenylethyl acrylate, 2-phenylethyl methacrylate, 2-(2′-hydroxy-5′-methacryloxyethylphenyl)-2H-benzotriazole or 2-(4-benzoyl-3-hydroxyphenoxy) ethyl acrylate.

The blue light absorber of the contact lens of the present disclosure can be but not limited to 4-(phenyldiazenyl) phenyl methacrylate.

The dye of the contact lens of the present disclosure can include anthocyanidin, beta-carotene, curcumin, luciferin, lutein, lycopene, phycobillin, phycoerythrim, phycocyanin, vitamin B2, zeaxanthin, photochromic dyes, thermochromic dyes, etc., and the derivatives thereof.

The beneficial agent of the contact lens of the present disclosure can include antibotic, bacteriostatic agent, antiviral agent, antifungal drugs, antiallergic agent, apomorphine, bromocriptine, dopamine receptor agonist, levodopa, quinpirole steroid, non-steroidal anti-inflammatory drug (NSAID), surfactants, miotics, enzyme inhibitor, anaesthetic, vasoconstrictor, nutrient, etc.

The antioxidant of the contact lens of the present disclosure can be vitamin A, vitamin C, vitamin E, uric acid, carotenoid, flavonoids, resveratrol, selenomethionine, etc.

The myopia control agent of the contact lens of the present disclosure can include a cycloplegic agent, a mydriatic agent, a selective/non-selective muscarinic receptor antagonist, etc., which has the effect of controlling, lessening, delaying or preventing the progression of myopia. For example, the ciliary muscle that controls the pupil can be relaxed and paralyzed by blocking the M-type muscarinic receptor of the parasympathetic nerve, so that can dilate pupils. The myopia control agent can be atropine ((3-endo)-8-methyl-8-azabicyclo[3.2.1]oct-3-yltropate), atropine sulphate, cyclopentolate (2-(dimethylamino)ethyl(1-hydroxycyclopentyl)(phenyl)acetate), cyclopentolate HCl, eucatropine (1,2,2,6-tetramethyl-4-piperidinylhydroxy(phenyl)acetate), homatropine ((3-endo)-8-methyl-8-azabicyclo[3.2.1]oct-3-ylhydroxy(phenyl)acetate), nuvenzepine, phenylephrine HCl, pirenzepine, raceanisodamine, rispenzepine, scopolamine ((1R,2R,4S,5S,7s)-9-methyl-3-oxa-9-azatricyclo[3.3.1.02,4]non-7-yl(2S)-3-hydroxy-2-phenylpropanoate), scopolamine HBr, telenzepine and tropicamide (N-ethyl-3-hydroxy-2-phenyl-N-(4-pyridinylmethyl)propanamide), etc., and the salts thereof.

The structural arrangement of the contact lens of the present disclosure can be a single-layer contact lens, and it also can be a contact lens having at least two layers. The color contact lens can be composed of two layers, such as the lens body layer and the color layer. The color contact lens can be composed of three layers, such as the lens body layer, the color layer and the anti-shedding protective layer. The color contact lens can be composed of four layers, such as the lens body layer, the first color layer, the second color layer and the anti-shedding protective layer. The color contact lens can be composed of five layers, such as the lens body layer, the first color layer, the second color layer, the third color layer and the anti-shedding protective layer. The color contact lens can be composed of another five of layers, such as the lens body layer, the first color layer, the separation layer, the second color layer and the anti-shedding protective layer.

The stable structure of the contact lens of the present disclosure can have a rotationally stable design, such as a hanged design of thickening for the bottom, a balanced design of thickening for the two sides, an asymmetrical balanced design, a stable design of thinning for the top and bottom, etc.

The functions of the contact lens of the present disclosure can be a myopia correction, a hyperopia correction, a presbyopia correction, an astigmatism correction, a myopia control, a corneal reshaping, etc.

The average transmittance of the contact lens of the present disclosure means the average value of the transmittances at the integer wavelength of every 1 nm. The average transmittance in the wavelength range from 400 nm to 700 nm is the average of the transmittances of every 1 nm wavelengths from 400 nm to 700 nm, that is, the average of the transmittances of 400 nm, 401 nm, 402 nm, 403 nm, and so on to 700 nm. The average transmittance in the wavelength range from 450 nm to 630 nm is the average of the transmittances of every 1 nm wavelengths from 450 nm to 630 nm, that is, the average of the transmittances of 450 nm, 451 nm, 452 nm, 453 nm, and so on to 630 nm.

The water content of the contact lens of the present disclosure is measured by hydrating the contact lens with the pure water and then calculating the water content based on the weight of the contact lens before and after the hydration according to ISO 18369-4 (2017). The formula is as follow: water content=(weight after the hydration−weight before the hydration)/weight after the hydration. The water content of the contact lens is affected by the type of the buffer solution used to soak the contact lens, so that the contact lens will be soaked in the buffer solution for at least 24 hours before the measurement.

The oxygen permeability of the contact lens of the present disclosure is calculated by hydrating the contact lens with the pure water according to ISO 18369-4 (2017). The oxygen permeability of the contact lens is affected by the type of the buffer solution used to soak the contact lens, so that the contact lens will be soaked in the buffer solution for at least 24 hours before the measurement.

The buffer solution of the present disclosure can include at least one, at least two, at least three or at least four humectants. The buffer solution can include at least one, at least two, at least three or at least four cooling agents. The buffer solution can include at least one, at least two, at least three or at least four eye-protecting components. The buffer solution can include at least one, at least two, at least three or at least four buffering agents. The buffer solution can include at least one, at least two, at least three or at least four antiseptics. The buffer solution can include at least one, at least two, at least three and at least four antioxidants. The buffer solution can include at least one, at least two, at least three or at least four chelating agents. The buffer solution can include at least one, at least two, at least three or at least four surfactants.

The humectant in the buffer solution of the present disclosure can include the glucan, PEG-40 hydrogenated castor oil, PEG-400, glycerin, allantoin, hyaluronic acid, ceramide, cholesterol, sea buckthorn fruit oil, γ-polyglutamic acid (γ-PGA), trehalose, alginic acid, hydroxypropyl methylcellulose (HPMC), 2-methacryloyloxyphosphorylcholine (MPC), etc., and the salts thereof.

The three-dimensional configuration of the glucan in the buffer solution of the present disclosure can be a D-form or an L-form. The glucan can include an alpha-glucan and a beta-glucan. The alpha-glucan can include alpha-1-3-glucan, alpha-1-4-glucan, alpha-1-6-glucan, alpha-1-3/1-4-glucan, alpha-1-4/1-6-glucan and alpha-1-3/1-6-glucan. The main chain of alpha-glucan can be 1,3 bonded, 1,4-bonded or 1,6-bonded, and the branch chain of alpha-glucan can be 1,3-bonded, 1,4-bonded or 1,6-bonded. The beta-glucan can include beta-1-3-glucan, beta-1-4-glucan, beta-1-6-glucan, beta-1-3/1-4-glucan, beta-1-4/1-6-glucan and beta-1-3/1-6-glucan. The main chain of beta-glucan can be 1,3 bonded, 1,4-bonded or 1,6-bonded, and the branch chain of beta-glucan can be 1,3-bonded, 1,4-bonded or 1,6-bonded. The glucan in the buffer solution can further include a beta-1-3/1-6-D-glucan in the polysaccharide of the lingzhi. The main chain of the beta-1-3/1-6-D-glucan is 1,3 bonded, and the branch chain of the beta-1-3/1-6-D-glucan is 1,6-bonded. The extraction source of the glucan in the buffer solution can be from the yeast, the cereal, the fungus, the plant, the bacteria, the algae, the lichen, etc. The extraction source being the yeast of the glucan in the buffer solution can be baker's yeast and brewer's yeast. The extraction source being the cereal of the glucan in the buffer solution can be the barley, the oatmeal, the wheat or the rice. The extraction source being the fungus of the glucan in the buffer solution can be the lingzhi, the white mushroom, the maitake mushroom, the mushroom and Aspergillus niger. The extraction source being the plant of the glucan in the buffer solution can be Aloe vera and Echinacea sp.

The molecular weight of the glucan in the buffer solution of the present disclosure means the number average molecular weight of the overall glucan in the buffer solution, and the formula thereof is: (Σ molecular weight of the glucan in the buffer solution)/the total number of molecule.

The particle size of the glucan in the buffer solution of the present disclosure means the median of the molecular diameter of the overall glucan in the buffer solution.

The cooling agent in the buffer solution of the present disclosure has the effect of making the affected portion feel cool. The cooling agent can include menthol, 2-isopropyl-N,2,3-trimethylbutyramide (WS-23), N-ethyl-p-menthane-3-carboxamide (WS-3), ethyl 3-(p-menthane-3-carboxamido)acetate (WS-5), (1R,2S,5R)—N-(4-methoxyphenyl)-p-menthanecarboxamide (WS-12), N-ethyl-2,2-diisopropylbutanamide (WS-27), N-(1,1-dimethyl-2-hydroxyethyl)-2,2-diethylbutanamide (WS-116), etc., and the salts thereof.

The eye-protecting component in the buffer solution of the present disclosure can include vitamin A, lutein, omega-3-fatty acid, anthocyanin, astaxanthin, carotene, blueberry, lecithin, or a combination thereof.

The buffering agent in the buffer solution of the present disclosure can be the buffer salt, such as citrate, acetate, phosphate, borate, sulfate, carbonate, nitrate, chloride, or a combination thereof. The cation of the buffer salt can be sodium, potassium, magnesium or calcium. The buffering agent further can be N-[tris(hydroxymethyl)methyl]-3-aminopropanesulfonic acid (TAPS), N,N-bis(2-hydroxyethyl)glycine (Bicine), tris(hydroxymethyl)aminomethane (Tris), N-tris(hydroxymethyl)methylaminoacetic acid (Tricine), 4-(2-hydroxyethyl)piperazine-1-ethanesulfonic acid (HEPES), N-tris(hydroxymethyl)methyl-2-aminoethanesulfonic acid (TES), 3-(N-morpholino)propanesulfonic acid (MOPS), pipeline-N,N′-bis(2-ethanesulfonic acid) (PIPES), cacodylate, 2-(N-morpholine)ethanesulfonic acid (MES), etc., and the salts thereof.

The antiseptic in the buffer solution of the present disclosure can include benzoic acid, sodium nitrite, calcium propionate, boric acid, sodium borate, methylparaben, DMDM Hydantoin (DMDMH), silver ion (Ag⁺), polyhexamethylene (PHMB), etc., and the salts thereof.

The antioxidant in the buffer solution of the present disclosure can include vitamin A, vitamin B, vitamin C, vitamin D, vitamin E, oxidized or reduced coenzyme Q10, glutathione, lipoic acid, carotene, lutein, dibutylhydroxytoluene (BHT), butylhydroxymethoxybenzene (BHA), propyl gallate (PG), tert-butylhydroquinone (TBHQ), etc., and the salts thereof.

The chelating agent in the buffer solution of the present disclosure can include ehtylenediamine (en), 2,2′-bipyridyl (bipy), 1, 10-phenanthroline (phen), oxalic acid, ethylenediaminetetraacetic acid (EDTA), 1,2-bis(dimethylarsine)benzene (diars), etc., and the salts thereof.

The surfactant in the buffer solution of the present disclosure can include poloxamer 407 (KP407), sodium dodecyl sulfate (SDS), cetyltrimethylammonium bromide (CTAB), trimethylstearylammonium chloride (STAC), potassium stearate, gum arabic, sodium alginate, etc., and the salts thereof.

The viscosity in the buffer solution of the present disclosure means the sticking property of the buffer solution, and it can represent the fluidity of the buffer solution. The viscosity is measured by a viscometer or a micro viscometer, and the rotating axis can be selected according to the measure volume and the measure rotation speed. The measure rotation speed can be selected from 0.01 rpm to 500 rpm, and the measure volume is at least 1 mL. The measurement details are in accordance with the operating manual of the used viscometer.

The buffer solution of the present disclosure can be used as the storage solution for the contact lens product, and also can be used as the eye wash, the moisturizer, the eye lotion, the eye drop and the contact lens cleaner.

The contact lens product of the present disclosure can be composed of the contact lens body, the buffer solution and the packaging, and the contact lens body is immersed in the buffer solution. The term of the contact lens of the present disclosure refers to the main body of the contact lens. The main body of the contact lens and the solution can include the humectant, the monomer, the UV absorber, the blue light absorber, the beneficial agent, the dye, the myopia control agent, the weak acid and its conjugate base salt, the weak base and its conjugate acid salt, the anionic agent, the cationic agent and other beneficial agents. The packaging of the present disclosure can be made of the plastic container and the aluminum foil upper cover. The plastic container can be made of polypropylene (PP), polystyrene (PS), polyethylene terephthalate (PET) or other plastic materials, and also can be made of the biodegradable plastic, such as, polylactic acid (PLA), polyhydroxyalkanoate (PHA) or other biodegradable plastics, etc. The biodegradable plastic also can be mixed with the biodegradable plasticizer to achieve the effects of the modification. The aluminum foil upper cover of the present disclosure can be the composite aluminum foil with the plastic coating.

Unless otherwise noted, the measurement environment of the present disclosure is at a temperature of 25° C. and a humidity of 50%.

According to the abovementioned embodiments, the specific examples are provided below and described in detail.

### 1st Comparative Example

The contact lens product of the 1st comparative example includes a contact lens and a buffer solution, and the contact lens is immersed in the buffer solution.

The contact lens of the 1st comparative example is a hydrogel contact lens, and the main monomer thereof is hydroxyethyl methacrylate. A composition of the contact lens includes a humectant, and the buffer solution includes a humectant. However, neither the humectant in the composition of the contact lens nor the humectant in the buffer solution includes a glucan.

The water content of the contact lens of the 1st comparative example is Cw, and Cw=54.07%.

### 1st Example

The contact lens product of the 1st example includes a contact lens and a buffer solution, and the contact lens is immersed in the buffer solution.

The contact lens of the 1st example is a hydrogel contact lens, and the main monomer thereof is hydroxyethyl methacrylate. A composition of the contact lens includes a humectant, and the buffer solution includes a humectant. The humectant in the composition of the contact lens includes a glucan, and the humectant in the buffer solution does not include a glucan.

Table 1 shows the composition and the parameter details of the contact lens and the buffer solution in the contact lens product of the 1st example. Pc is the weight percentage of the glucan in the composition of the contact lens, MWc is the molecular weight of the glucan in the composition of the contact lens, PSc is the particle size of the glucan in the composition of the contact lens, Cw is the water content of the contact lens, Dc is the diameter of the contact lens, T4070 is the average transmittance of the contact lens in a wavelength range from 400 nm to 700 nm, T4563 is the average transmittance of the contact lens in a wavelength range from 450 nm to 630 nm, Pb is the weight percentage of the glucan in the buffer solution, MWb is the molecular weight of the glucan in the buffer solution, PSb is the particle size of the glucan in the buffer solution, VI is the viscosity of the buffer solution, pHb is the pH value of the buffer solution, and Ob is the osmotic pressure of the buffer solution.

TABLE 1
Contact lens

| | |
|---|---|
| Humectant | Beta-1-3/1-6-D-glucan |
| Pc (%) | 0.009 |
| MWc (kDa) | — |
| PSc (nm) | — |
| Cw (%) | 55.8 |
| Dc (mm) | 14.15 |
| T4070 (%) | 92.50 |
| T4563 (%) | 92.63 |
| Contact lens type | Hydrogel contact lens |
| Main monomer | Hydroxyethyl methacrylate (HEMA) |

Buffer solution

| | |
|---|---|
| Humectant | — |
| Pb (%) | — |
| MWb (kDa) | — |
| PSb (nm) | — |
| VI (Pa · s) | — |
| pHb | — |
| Ob (Osmol/kg · H₂O) | — |

The parameter definitions of each of the following examples are the same as the parameter definitions in Table 1 of the 1st example, and it will not be described hereafter.

### 2nd Example

The contact lens product of the 2nd example includes a contact lens and a buffer solution, and the contact lens is immersed in the buffer solution.

The contact lens of the 2nd example is a hydrogel contact lens, and the main monomer thereof is hydroxyethyl methacrylate. A composition of the contact lens includes a humectant, and the buffer solution includes a humectant. The humectant in the composition of the contact lens includes a glucan, and the humectant in the buffer solution does not include a glucan.

Table 2 shows the composition and the parameter details of the contact lens and the buffer solution in the contact lens product of the 2nd example.

TABLE 2
Contact lens

| | |
|---|---|
| Humectant | Beta-1-3/1-6-D-glucan |
| Pc (%) | 0.03 |
| MWc (kDa) | — |
| PSc (nm) | — |
| Cw (%) | 55.4 |
| Dc (mm) | 14.13 |
| T4070 (%) | 92.28 |
| T4563 (%) | 92.42 |
| Contact lens type | Hydrogel contact lens |
| Main monomer | Hydroxyethyl methacrylate (HEMA) |

Buffer solution

| | |
|---|---|
| Humectant | — |
| Pb (%) | — |
| MWb (kDa) | — |
| PSb (nm) | — |
| VI (Pa · s) | — |
| pHb | — |
| Ob (Osmol/kg · H₂O) | — |

### 3rd Example

The contact lens product of the 3rd example includes a contact lens and a buffer solution, and the contact lens is immersed in the buffer solution.

The contact lens of the 3rd example is a hydrogel contact lens, and the main monomer thereof is hydroxyethyl methacrylate. A composition of the contact lens includes a humectant, and the buffer solution includes a humectant. The humectant in the composition of the contact lens does not include a glucan, and the humectant in the buffer solution includes a glucan.

Table 3 shows the composition and the parameter details of the contact lens and the buffer solution in the contact lens product of the 3rd example.

TABLE 3
Contact lens

| | |
|---|---|
| Humectant | — |
| Pc (%) | — |
| MWc (kDa) | — |
| PSc (nm) | — |
| Cw (%) | 55.9 |
| Dc (mm) | 14.1 |
| T4070 (%) | 92.70 |
| T4563 (%) | 92.84 |
| Contact lens type | Hydrogel contact lens |
| Main monomer | Hydroxyethyl methacrylate (HEMA) |

Buffer solution

| | |
|---|---|
| Humectant | Beta-1-3/1-6-D-glucan |
| Pb (%) | 0.006 |
| MWb (kDa) | 160~650 |
| PSb (nm) | 1800~2000 |
| VI (Pa · s) | 1.05 |
| pHb | 7.42 |
| Ob (Osmol/kg · H₂O) | 0.334 |

### 4th Example

The contact lens product of the 4th example includes a contact lens and a buffer solution, and the contact lens is immersed in the buffer solution.

The contact lens of the 4th example is a hydrogel contact lens, and the main monomer thereof is hydroxyethyl methacrylate. A composition of the contact lens includes a humectant, and the buffer solution includes a humectant. Both the humectant in the composition of the contact lens and the humectant in the buffer solution include a glucan.

Table 4 shows the composition and the parameter details of the contact lens and the buffer solution in the contact lens product of the 4th example.

TABLE 4
Contact lens

| | |
|---|---|
| Humectant | Beta-1-3/1-6-D-glucan |
| Pc (%) | 0.009 |
| MWc (kDa) | 160~650 |
| PSc (nm) | 1800~2000 |
| Cw (%) | 54.4 |
| Dc (mm) | 14.13 |
| T4070 (%) | 92.64 |
| T4563 (%) | 92.83 |
| Contact lens type | Hydrogel contact lens |
| Main monomer | Hydroxyethyl methacrylate (HEMA) |

Buffer solution

| | |
|---|---|
| Humectant | Beta-1-3/1-6-D-glucan |
| Pb (%) | 0.006 |
| MWb (kDa) | 160~650 |
| PSb (nm) | 1800~2000 |
| VI (Pa · s) | 1.05 |
| pHb | 7.42 |
| Ob (Osmol/kg · H₂O) | 0.334 |

### 5th Example

The contact lens product of the 5th example includes a contact lens and a buffer solution, and the contact lens is immersed in the buffer solution.

The contact lens of the 5th example is a hydrogel contact lens, and the main monomer thereof is hydroxyethyl methacrylate. A composition of the contact lens includes a humectant, and the buffer solution includes a humectant. Both the humectant in the composition of the contact lens and the humectant in the buffer solution include a glucan.

Table 5 shows the composition and the parameter details of the contact lens and the buffer solution in the contact lens product of the 5th example.

TABLE 5
Contact lens

| | |
|---|---|
| Humectant | Beta-1-3/1-6-D-glucan |
| Pc (%) | 0.015 |
| MWc (kDa) | 160~650 |
| PSc (nm) | 1800~2000 |
| Cw (%) | 55.2 |
| Dc (mm) | 14.13 |
| T4070 (%) | 91.09 |
| T4563 (%) | 91.20 |
| Contact lens type | Hydrogel contact lens |
| Main monomer | Hydroxyethyl methacrylate (HEMA) |

Buffer solution

| | |
|---|---|
| Humectant | Beta-1-3/1-6-D-glucan |
| Pb (%) | 0.01 |
| MWb (kDa) | 160~650 |
| PSb (nm) | 1800~2000 |
| VI (Pa · s) | 1.01 |
| pHb | 7.42 |
| Ob (Osmol/kg · H₂O) | 0.296 |

The foregoing description, for purpose of explanation, has been described with reference to specific embodiments. It is to be noted that Tables show different data of the different embodiments; however, the data of the different embodiments are obtained from experiments. The embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the present disclosure to the precise forms disclosed. Many modifications and variations are possible in view of the above teachings.

What is claimed is:

1. A contact lens comprising a composition of the contact lens, the composition of the contact lens comprising:
a humectant comprising a glucan, and the glucan in the composition of the contact lens comprising an 1-3/1-6-glucan;
wherein a weight percentage of the glucan in the composition of the contact lens is Pc, an average transmittance of the contact lens in a wavelength range from 400 nm to 700 nm is T4070, and the following conditions are satisfied:
0.001%≤Pc≤1.00%; and
90%≤T4070.

2. The contact lens of claim 1, wherein the weight percentage of the glucan in the composition of the contact lens is Pc, and the following condition is satisfied:
0.003%≤Pc≤0.10%.

3. The contact lens of claim 1, wherein a molecular weight of the glucan in the composition of the contact lens is MWc, and the following condition is satisfied:
10 kDa≤MWc≤2000 kDa.

4. The contact lens of claim 1, wherein a particle size of the glucan in the composition of the contact lens is PSc, and the following condition is satisfied:
50 nm≤PSc≤5000 nm.

5. The contact lens of claim 1, wherein a diameter of the contact lens is Dc, and the following condition is satisfied:
13.8 mm≤Dc≤14.5 mm.

6. The contact lens of claim 5, wherein the contact lens is a hydrogel contact lens, and a main monomer of the contact lens is hydroxyethyl methacrylate.

7. The contact lens of claim 6, wherein the composition of the contact lens further comprises:
an UV absorber or a blue light absorber;
a dye;
a beneficial agent;
an antioxidant; and
a myopia control agent.

8. A contact lens product, comprising:
the contact lens of claim 1; and
a buffer solution, wherein the contact lens is immersed in the buffer solution.

9. The contact lens product of claim 8, wherein the buffer solution comprises a humectant, the humectant in the buffer solution comprises a glucan, and the glucan in the buffer solution comprises a beta-glucan;
wherein a weight percentage of the glucan in the buffer solution is Pb, and the following condition is satisfied:
0.0003%≤Pb≤1.00%.

10. The contact lens product of claim 9, wherein a molecular weight of the glucan in the buffer solution is MWb, and the following condition is satisfied:
50 kDa≤MWb≤1500 kDa.

11. The contact lens product of claim 10, wherein a particle size of the glucan in the buffer solution is PSb, and the following condition is satisfied:
500 nm≤PSb≤4000 nm.

12. The contact lens product of claim 11, wherein a water content of the contact lens is Cw, and the following condition is satisfied:
54.5%≤Cw.

13. A contact lens comprising a composition of the contact lens, the composition of the contact lens comprising:
a humectant comprising a glucan, and the glucan in the composition of the contact lens comprising a beta-glucan;
wherein a weight percentage of the glucan in the composition of the contact lens is Pc, an average transmittance of the contact lens in a wavelength range from 450 nm to 630 nm is T4563, and the following conditions are satisfied:
0.0001%≤Pc; and
90%≤T4563.

14. The contact lens of claim 13, wherein the beta-glucan in the composition of the contact lens comprises an 1-3/1-6-glucan.

15. The contact lens of claim 14, wherein the weight percentage of the glucan in the composition of the contact lens is Pc, and the following condition is satisfied:
0.0005%≤Pc≤5.00%.

16. The contact lens of claim 15, wherein a molecular weight of the glucan in the composition of the contact lens is MWc, and the following condition is satisfied:
30 kDa≤MWc≤1800 kDa.

17. The contact lens of claim 16, wherein a particle size of the glucan in the composition of the contact lens is PSc, and the following condition is satisfied:
500 nm≤PSc≤4000 nm.

18. A contact lens product, comprising:
the contact lens of claim 13.

19. A contact lens product, comprising:
a contact lens; and
a buffer solution, wherein the contact lens is immersed in the buffer solution;
wherein the buffer solution comprises a humectant, the humectant in the buffer solution comprises a glucan, and the glucan in the buffer solution comprises a beta-glucan;
wherein a weight percentage of the glucan in the buffer solution is Pb, and the following condition is satisfied:
0.0001%≤Pb≤5.00%.

20. The contact lens product of claim 19, wherein the weight percentage of the glucan in the buffer solution is Pb, and the following condition is satisfied:
0.001%≤Pb≤0.30%.

21. The contact lens product of claim 20, wherein the beta-glucan in the buffer solution comprises an 1-3/1-6-glucan.

22. The contact lens product of claim 19, wherein a composition of the contact lens comprises a humectant, and the humectant in the composition of the contact lens comprises a beta-glucan.

23. The contact lens product of claim 19, wherein a molecular weight of the glucan in the buffer solution is MWb, and the following condition is satisfied:
10 kDa≤MWb≤2000 kDa.

24. The contact lens product of claim 23, wherein a particle size of the glucan in the buffer solution is PSb, and the following condition is satisfied:
50 nm≤PSb≤5000 nm.

25. The contact lens product of claim 24, wherein a viscosity of the buffer solution is VI, and the following condition is satisfied:
VI≤10 Pa·s.

26. The contact lens product of claim 25, wherein a pH value of the buffer solution is pHb, and the following condition is satisfied:
7.3≤pHb≤7.5.

27. The contact lens product of claim 26, wherein an osmotic pressure of the buffer solution is Ob, and the following condition is satisfied:
0.280 Osmol/kg·H₂O≤Ob≤0.350 Osmol/kg·H₂O.

28. The contact lens product of claim 19, wherein:
a composition of the contact lens comprises a humectant, and the humectant in the composition of the contact lens comprises a glucan; and
a weight percentage of the glucan in the composition of the contact lens is Pc, an average transmittance of the contact lens in a wavelength range from 400 nm to 700 nm is T4070, an average transmittance of the contact lens in a wavelength range from 450 nm to 630 nm is T4563, a molecular weight of the glucan in the composition of the contact lens is MWc, a particle size of the glucan in the composition of the contact lens is PSc, a diameter of the contact lens is Dc, the weight percentage of the glucan in the buffer solution is Pb, a molecular weight of the glucan in the buffer solution is MWb, a particle size of the glucan in the buffer solution is PSb, a viscosity of the buffer solution is VI, a pH value of the buffer solution is pHb, an osmotic pressure of the buffer solution is Ob, and the following conditions are satisfied:
0.008%≤Pc≤0.013%;
92.5%≤T4070;
92.5%≤T4563;
150 kDa≤MWc≤700 kDa;
1600 nm≤PSc≤2100 nm;
14.0 mm≤Dc≤14.2 mm;
0.0055%≤Pb≤0.008%;
150 kDa≤MWb≤700 kDa;
1600 nm≤PSb≤2100 nm;
0.9 Pa·s≤VI≤1.1 Pa·s;
7.35≤pHb≤7.45; and
0.295 Osmol/kg·H₂O≤Ob≤0.305 Osmol/kg·H₂O.
